(19) United States
(12) Patent Application Publication
(10) Pub. No.: US 2016/0095842 A1
(43) Pub. Date: Apr. 7, 2016

(54) COMBINATION THERAPY CONTAINING A PI3K-ALPHA INHIBITOR AND FGFR KINASE INHIBITOR FOR TREATING CANCER

(71) Applicants: Christine FRITSCH, Basel (CH); Christian CHATENAY-RIVAUDAY, Basel (CH); Johannes ROESEL, Basel (CH); Markus WARTMANN, Basel (CH); Diana GRAUS PORTA, Basel (CH)

(72) Inventors: Christine Fritsch, STEINBACH (FR); Christian Chatenay-Rivauday, Saint Louis (FR); Johannes Roesel, Riehen (CH); Markus Wartmann, Riehen (CH); Diana Graus Porta, Basel (CH)

(21) Appl. No.: 14/892,082

(22) PCT Filed: May 28, 2014
(86) PCT No.: PCT/IB2014/061789

§ 371 Date: Nov. 18, 2015

Related U.S. Application Data
(60) Provisional application No. 61/829,317, filed on May 31, 2013.

(51) Int. Cl. A61K 31/4439; C12Q 1/68; A61K 31/506

(52) U.S. Cl. CPC A61K 31/4439; A61K 31/506; C12Q 1/6886; C12Q 2600/156; C12Q 2600/106

(57) ABSTRACT

A pharmaceutical combination comprising an alpha-isoform specific phosphatidylinositol 3-kinase inhibitor compound of formula (I) or a pharmaceutically acceptable salt thereof and a fibroblast growth factor receptor (FGFR) kinase inhibitor, particularly for simultaneous, separate or sequential use for the treatment of a cancer; use of such pharmaceutical combination for the preparation of a medicament for the treatment of a cancer; use of such pharmaceutical combination for the treatment a cancer; a method for treating a cancer comprising administering to a subject having said cancer a jointly therapeutically effective amount of such combination; and related pharmaceutical compositions or kits thereof.

### FIELD OF THE INVENTION

A pharmaceutical combination comprising an alpha-isoform specific phosphatidylinositol 3-kinase inhibitor compound of formula (I), as described herein, or a pharmaceutically acceptable salt thereof and a fibroblast growth factor receptor (FGFR) kinase inhibitor, particularly for simultaneous, separate or sequential use for the treatment of a cancer; use of such pharmaceutical combination for the preparation of a medicament for the treatment of a cancer; use of such pharmaceutical combination for the treatment of a cancer; a method for treating a cancer comprising administering to a subject having said cancer a jointly therapeutically effective amount of such combination; and related pharmaceutical compositions or kits thereof.

### BACKGROUND OF THE INVENTION

The PI3K/Akt/mTOR pathway is an important, tightly regulated survival pathway for the normal cell. Phosphatidylinositol 3-kinases (PI3Ks) are widely expressed lipid kinases that catalyze the transfer of phosphate to the D-3′ position of inositol lipids to produce phosphoinositol-3-phosphate (PIP), phosphoinositol-3,4-diphosphate (PIP₂) and phosphoinositol-3,4,5-triphosphate (PIP₃). These products of the PI3K-catalyzed reactions act as second messengers and have central roles in key cellular processes, including cell growth, differentiation, mobility, proliferation and survival.

Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class 1B sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)).

In many cases, PIP₂ and PIP₃ recruit AKT to the plasma membrane where it acts as a nodal point for many intracellular signaling pathways important for growth and survival (Fantl et al., Cell 69:413-423(1992); Bader et al., Nature Rev. Cancer 5:921 (2005); Vivanco and Sawyer, Nature Rev. Cancer 2:489 (2002)). Aberrant regulation of PI3K, which often increases survival through AKT activation, is one of the most prevalent events in human cancer and has been shown to occur at multiple levels. In some tumors, the genes for the p110α isoform (PIK3CA) and for AKT are amplified and increased protein expression of their gene products has been demonstrated in several human cancers. Further, somatic missense mutations in PIK3CA that activate downstream signaling pathways have been described at significant frequencies in a wide diversity of human cancers (Kang at el., Proc. Natl. Acad. Sci. USA 102:802 (2005); Samuels et al., Science 304:554 (2004); Samuels et al., Cancer Cell 7:561-573 (2005)). Thus, highly specific inhibitors of the alpha-isoform of PI3K are particularly valuable in the treatment of cancer.

Fibroblast growth factor receptors (FGFRs) comprise a subfamily of receptor tyrosine kinases that are master regulators of a broad spectrum of biological activities, including development, metabolism, angiogenesis, apoptosis, proliferation and migration. Several distinct membrane FGFRs with tyrosine kinase activity have been identified in vertebrates: FGFR1 (═CD331, see also Fibroblast growth factor receptor 1); FGFR2 (═CD332, see also Fibroblast growth factor receptor 2); FGFR3 (═CD333, see also Fibroblast growth factor receptor 3); FGFR4 (═CD334, see also Fibroblast growth factor receptor 4); and FGFR6. Due to their broad impact, FGFRs are highly regulated and normally only basally active.

Epidemiological studies have reported genetic alterations and/or abnormal expression of FGFs/FGFRs in human cancers: translocation and fusion of FGFR1 to other genes resulting in constitutive activation of FGFR1 kinase is responsible for 8p11 myeloproliferative disorder (MacDonald D and Cross N C, Pathobiology 74:81-8 (2007)). Gene amplification and protein over-expression have been reported for FGFR1, FGFR2 and FGFR4 in breast tumors (Adnane J et al., Oncogene 6:659-63 (1991); Jaakkola S et al., Int. J. Cancer 54:378-82 (1993); Penault-Llorca F et al., Int. J. Cancer 61: 170-6 (1995); Reis-Filho J S et al., Clin. Cancer Res. 12:6652-62 (2006)). Somatic activating mutations of FGFR2 are known in gastric (Jang J H et al., Cancer Res. 61:3541-3 (2001)) and endometrial cancers (Pollock P M et al., Oncogene (May 21, 2007)). Recurrent chromosomal translocations of 4p16 into the immunoglobuling heavy chain switch region at 14q32 result in deregulated over-expression of FGFR3 in multiple myeloma (Chesi M et al., Nature Genetics 16:260-264 (1997); Chesi M et al., Blood 97:729-736 (2001)) and somatic mutations in specific domains of FGFR3 leading to ligand-independent constitutive activation of the receptor have been identified in urinary bladder carcinomas and multiple myelomas (Cappellen D et al., Nature Genetics 23:18-20 (1999); Billerey C et al., Am. J. Pathol. 158(6):1955-9 (2001); van Rhijn B W G et al., Eur. J. Hum. Genet. 10: 819-824 (2002); Ronchetti C et al., Oncogene 20: 3553-3562 (2001)).

In spite of numerous treatment options for cancer patients, there remains a need for effective and safe therapeutic agents and a need for their preferential use in combination therapy. The compounds of formula (I) are novel compounds that selectively inhibit the alpha (a) isoform of PI3K. It has been surprisingly found that the compound of formula (I) has a strong beneficial synergistic interaction and improved anti-proliferative activity when used in combination with FGFR kinase inhibitors. It is therefore an object of the present invention to provide for a medicament to improve treatment of cancer.

### SUMMARY OF THE INVENTION

The present invention provides a pharmaceutical combination comprising (a) an alpha-isoform specific phosphatidylinositol 3-kinase inhibitor (PI3K) compound of formula (I),

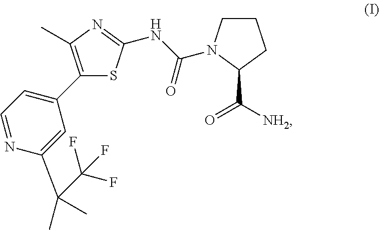 [SMILES: Cc1nc(NC(=O)N2CCC[C@H]2C(N)=O)sc1-c1ccnc(C(C)(C)C(F)(F)F)c1]

or a pharmaceutically acceptable salt thereof; and (b) an fibroblast growth factor receptor (FGFR) kinase inhibitor, or a pharmaceutically acceptable salt thereof. In one embodiment, such pharmaceutical combination is for simultaneous, separate or sequential use for the treatment of a cancer.

In a further embodiment, the present invention provides the use of a pharmaceutical combination comprising a compound of formula (I) or a pharmaceutically acceptable salt thereof and an FGFR kinase inhibitor, or a pharmaceutically acceptable salt thereof, for the preparation of a medicament for the treatment of a cancer.

In a further embodiment, the present invention provides the use of a pharmaceutical combination comprising a compound of formula (I) or a pharmaceutically acceptable salt thereof and an FGFR kinase inhibitor, or a pharmaceutically acceptable salt thereof, for the treatment of a cancer.

In a further embodiment, the present invention provides a method of treating a cancer comprising administering to a subject, especially a human, having said cancer a jointly therapeutically effective amount of a compound of formula (I) or a pharmaceutically acceptable salt thereof and an FGFR kinase inhibitor, or a pharmaceutically acceptable salt thereof.

In a further embodiment, the present invention provides a pharmaceutical composition comprising a compound of formula (I) or a pharmaceutically acceptable salt thereof and an FGFR kinase inhibitor, or a pharmaceutically acceptable salt thereof for the treatment of a cancer. Optionally and preferably the pharmaceutical composition further comprises a pharmaceutically acceptable excipitent.

In a further embodiment, the present invention provides the use of compound of formula (I) or a pharmaceutically acceptable salt thereof for the manufacture of a medicament to be used in combination with at least one FGFR kinase inhibitor, or a pharmaceutically acceptable salt thereof.

In a further embodiment, the present invention provides the use of at least one FGFR kinase inhibitor, or a pharmaceutically acceptable salt thereof, for the manufacture of a medicament to be used in combination with compound of formula (I) or a pharmaceutically acceptable salt thereof.

In a further embodiment, the present invention provides the combination of compound of formula (I) and at least one FGFR kinase inhibitor, or a pharmaceutically acceptable salt thereof, for use in the treatment of cancer.

In a further embodiment, the present invention further relates to a kit comprising a compound of formula (I) or a pharmaceutically acceptable salt thereof, and a package insert or label providing instruction for treating a cancer by co-administering at least one FGFR kinase inhibitor, or a pharmaceutically acceptable salt thereof.

### DETAILED DESCRIPTION OF THE FIGURES

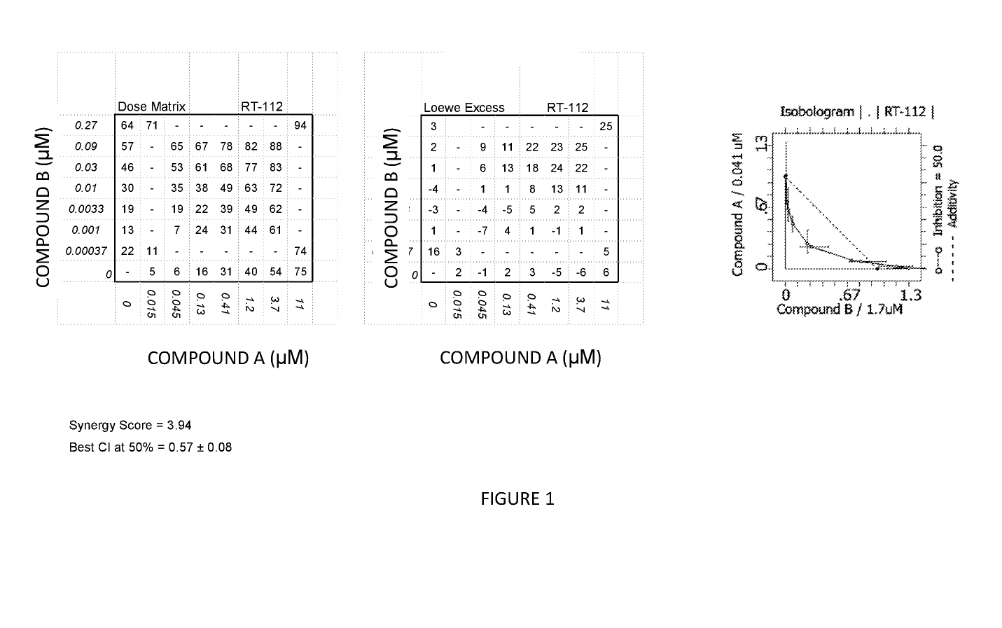

FIG. 1 illustrates the effects of combining Compound A and Compound B doses on proliferation of RT112 urothelial carcinoma cells. In the experiments described in Example 1, the combination of Compound A and Compound B produced a synergistic interaction in RT112 urothelial carcinoma cells.

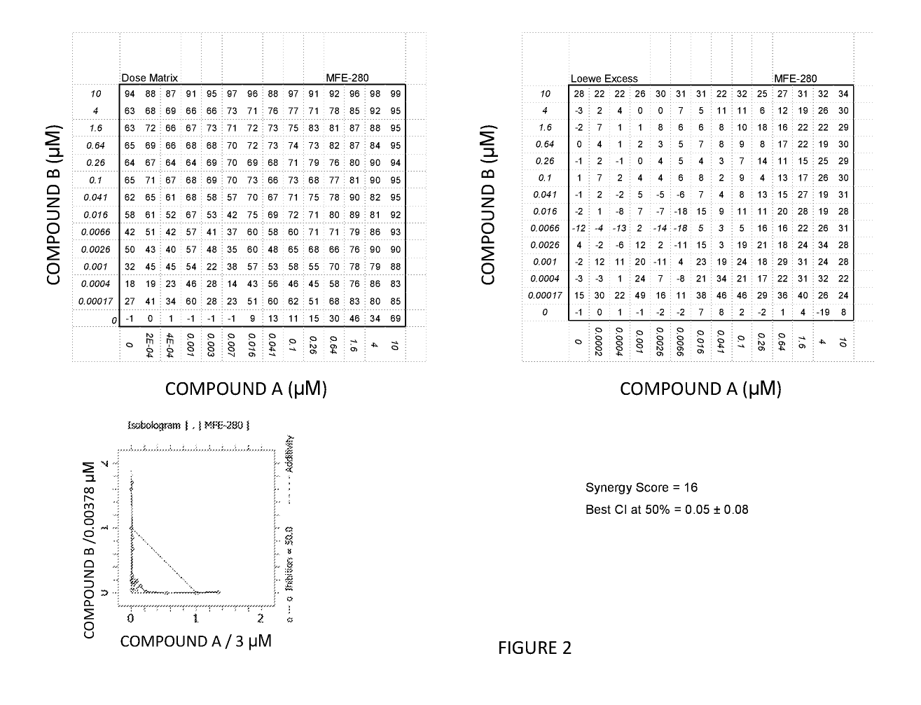

FIG. 2 illustrates the effects of combining Compound A and Compound B doses on proliferation of MFE280 endometrial cancer cells. In the experiments described in Example 1, the combination of Compound A and Compound B produced a synergistic interaction in MFE280 endometrial cancer cells.

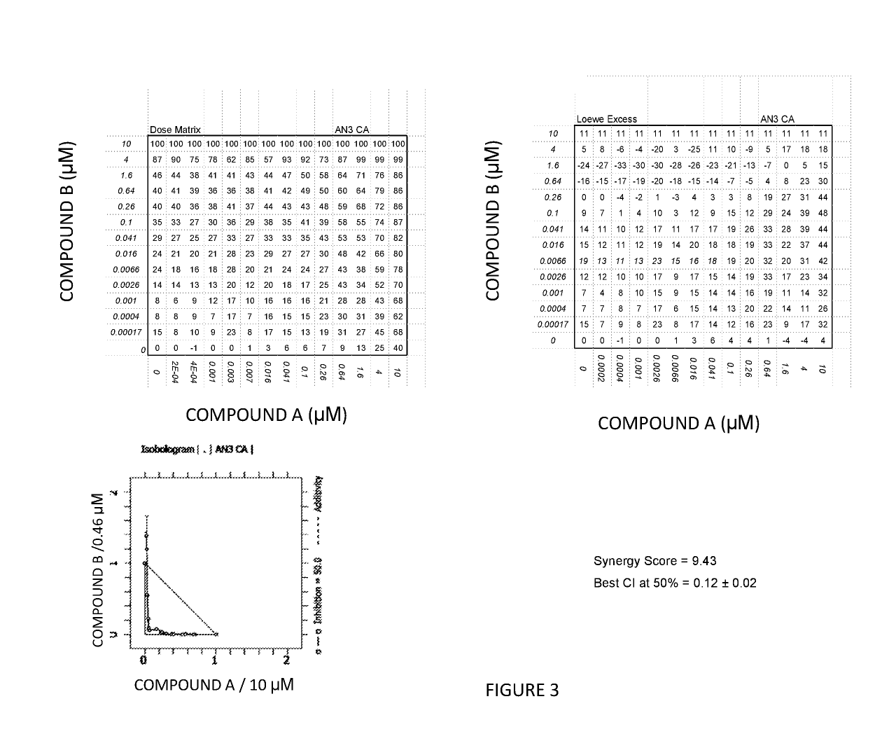

FIG. 3 illustrates the effects of combining Compound A and Compound B doses on proliferation of AN3CA endometrial cancer cells. In the experiments described in Example 1, the combination of Compound A and Compound B produced a synergistic interaction in AN3CA endometrial cancer cells.

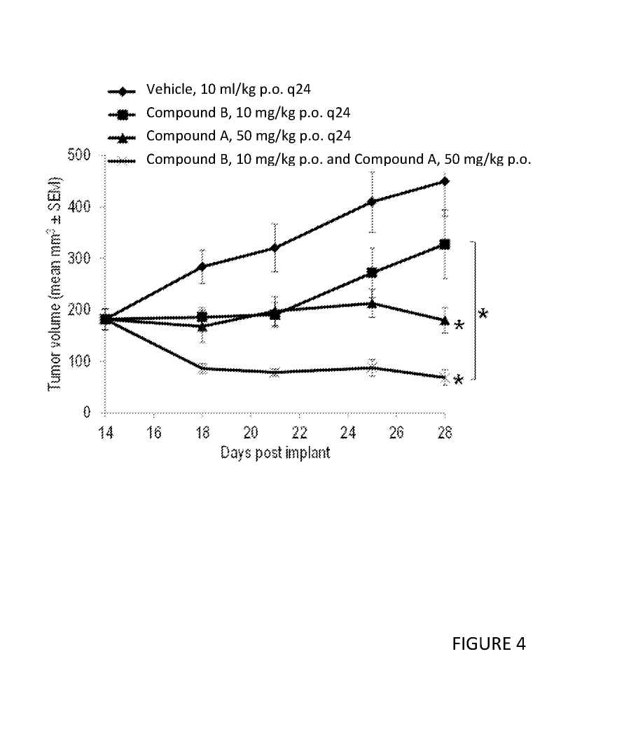

FIG. 4 shows the antitumor activity of 50 mg/kg, p.o. Compound A in combination with 10 mg/kg p.o. Compound B against RT112 urinary bladder carcinoma. Female HsdNpa:Athymic nude mice bearing subcutaneous (s.c.) RT112 urinary bladder carcinoma are treated p.o. either with the alpha-isoform specific PI3K inhibitor Compound A, the FGFR kinase inhibitor Compound B, or a combination thereof or a vehicle control at the indicated dose and schedule.

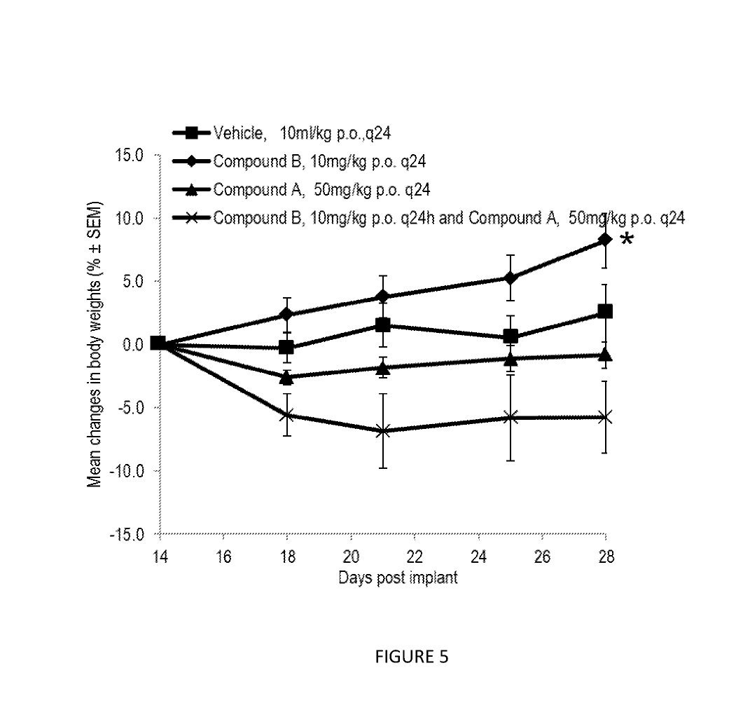

FIG. 5 shows the average body weight change for female HsdNpa:Athymic nude mice bearing s.c. RT112 urinary bladder carcinoma at Day 0 to Day 14 of treatment with the alpha-isoform specific PI3K inhibitor Compound A, the FGFR kinase inhibitor Compound B, or a combination thereof or a vehicle control at the indicated dose and schedule.

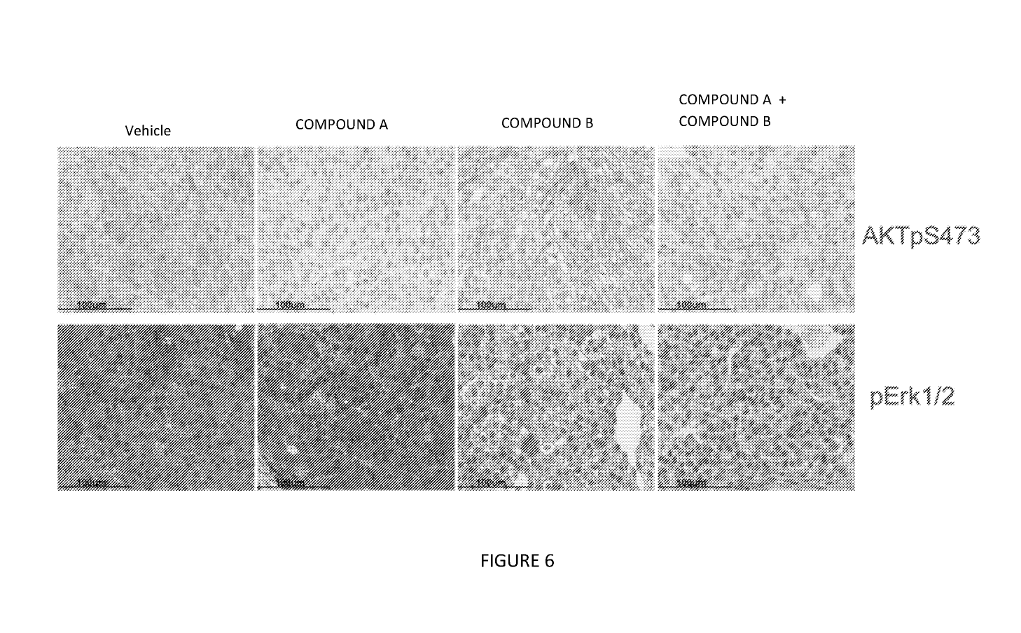

FIG. 6 shows the levels of resulting inhibition of pAKT (or AKTpS473) and/or pErk1/2 in RT112 urinary bladder carcinoma cells treated with Compound A alone, Compound B alone and the combination of Compound A and Compound B.

As shown in FIG. 6, the immunohistochemistry data shows that treatment with Compound A significantly inhibits pAKT, that treatment with Compound B significantly inhibits pErk1/2 and that treatment with the combination of Compound A and Compound B results in inhibition of both, pAKT and pErk1/2.

### DETAILED DESCRIPTION OF THE INVENTION

The present invention provides a pharmaceutical combination comprising (a) an alpha-isoform specific phosphatidylinositol 3-kinase (PI3K) inhibitor compound of formula (I), as defined below, or a pharmaceutically acceptable salt thereof; and (b) an fibroblast growth factor receptor (FGFR) kinase inhibitor, or a pharmaceutically acceptable salt thereof. Such combination may be for simultaneous, separate or sequential use for the treatment of a cancer.

The following general definitions are provided to better understand the invention:

“Combination” refers to either a fixed combination in one dosage unit form, or a non-fixed combination, such as kit of parts, for the combined administration where a compound of the formula (I) and a combination partner (e.g. another therapeutic agent or active ingredient or drug as explained below) may be administered independently at the same time or separately within time intervals, especially where these time intervals allow that the combination partners show a cooperative, e.g. synergistic, effect and/or jointly therapeutic effect. This also applies to cocktail therapy, e.g., the administration of three or more active ingredients.

“Fixed combination” means that two or more active ingredients, e.g. a compound of formula (I) and a combination partner, are administered to a subject simultaneously in the form of a single entity or dosage form.

“Non-fixed combination” means that two or more active ingredients, e.g. a compound of formula (I) and a combination partner, are simultaneously, concurrently or sequentially administered to a patient as separate entities with no specific time limits, wherein such administration provides therapeutically effective levels of said compounds in the body of the patient. Preferably the time intervals allow that the combination partners show a cooperative, e.g. synergistic, effect and/or jointly therapeutic effect.

“Combined administration” or “co-administer” refers to administration of two or more active ingredients to a single subject in need thereof (e.g. a patient), and includes treatment regimens in which all active ingredients are not necessarily administered by the same route of administration or at the same time.

“Treat”, “treating, or “treatment” refers to prophylactic or therapeutic treatment (including but not limited to palliative, curing, symptom-alleviating, symptom-reducing) or the delay of progression of a cancer. The term “prophylactic” refers to the prevention of the onset or recurrence of a cancer. The term “delay of progression” refers to administration of the pharmaceutical combination to patients being in a pre-stage or in an early phase of a cancer, to be treated, a pre-form of the corresponding cancer is diagnosed and/or in a patient diagnosed with a condition under which it is likely that a corresponding cancer will develop.

“Pharmaceutical composition” or “medicament” refers to a mixture or solution containing at least one active ingredient to be administered to a subject, e.g., a human.

“Pharmaceutically acceptable” refers to those compounds, materials, compositions and/or dosage forms, which are, within the scope of sound medical judgment, suitable for contact with the tissues of subjects, especially humans, without excessive toxicity, irritation, allergic response and other problem complications commensurate with a reasonable benefit/risk ratio.

“Therapeutically effective” preferably relates to an amount of an active ingredient that is therapeutically or in a broader sense also prophylactically effective against the progression of a cancer.

“Jointly therapeutically effective” means that the two or more active ingredients may be given simultaneously (in one dosage form or multiple dosage forms) or separately (in a chronologically staggered manner, especially a sequence-specific manner) in such time intervals that they prefer, in the subject, especially human, to be treated, and still show a (preferably synergistic) interaction. Whether this is the case can, inter alia, be determined by following the blood levels, showing that both compounds are present in the blood of the human to be treated at least during certain time intervals.

“Single pharmaceutical composition” refers to a single dosage form formulated to deliver therapeutically effective amounts of both active ingredients to a patient. The single dosage form is designed to deliver an effective amount of each of the agents, along with any pharmaceutically acceptable carriers. In some embodiments, the dosage form is a tablet, capsule, pill, or a patch. In other embodiments, the dosage form is a solution or a suspension.

“Carrier” or “pharmaceutically acceptable carrier” includes any and all solvents, dispersion media, coatings, surfactants, antioxidants, preservatives, isotonic agents, absorption delaying agents, drug stabilizers, diluents, binders, excipients, disintegrants, lubricants, dyes, sweeteners, flavoring agents, and the like and/or combinations thereof, as would be known to those skilled in the art (see, for example, Remington's Pharmaceutical Sciences, 18^{th} Ed. Mack Printing Company, 1990, pp. 1289-1329).

“Dose range” refers to an upper and a lower limit of an acceptable variation of the amount of active ingredient specified. Typically, a dose of the agent in any amount within the specified range can be administered to patients undergoing treatment.

“Subject” or “patient” refers to a warm-blooded animal, including for example primates, humans, dogs, cows, horses, pigs, sheep, goats, cats, mice, rabbits, rats, and transgenic non-human animals. In certain embodiments, the subject or patient is a human, e.g., a human suffering from, at risk of suffering from, or potentially capable of suffering from a brain tumor disease. Particularly preferred, the subject is human.

A subject is “in need of” a treatment if such subject would benefit biologically, medically or in quality of life from such treatment.

“A”, “an”, “the” and similar terms used in the context of the present invention (especially in the context of the claims) are to be construed to cover both the singular and plural unless otherwise indicated herein or clearly contradicted by the context.

The terms “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value

In the present invention, the pharmaceutical combination comprises the alpha-isoform specific phosphatidylinositol 3-kinase (PI3K) inhibitor compound of formula (I)

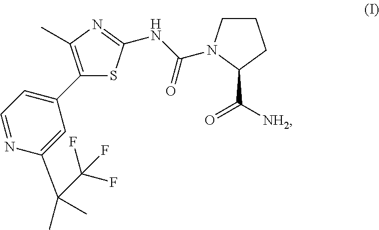 [SMILES: Cc1nc(NC(=O)N2CCC[C@H]2C(N)=O)sc1-c1ccnc(C(C)(C)C(F)(F)F)c1]

(also referred to as “Compound A”) or any pharmaceutically acceptable salt thereof. The compound of formula (I) is known by the chemical name (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide). The compound of formula (I) and its pharmaceutically acceptable salts, their preparation and suitable pharmaceutical formulations containing the same are described in WO 2010/029082, which is hereby incorporated by reference in its entirety. The synthesis of the compound of formula (I) is described in WO2010/029082 as Example 15.

As shown in WO2010/029082, the compound of formula (I) has been found to have significant inhibitory activity for the alpha-isoform of phosphatidylinositol 3-kinases (or PI3K). The compound of formula (I) has advantageous pharmacological properties as a PI3K inhibitor and shows a high selectivity for the PI3-kinase alpha isoform as compared to the beta and/or delta and/or gamma isoforms.

The compound of formula (I) may be incorporated in the pharmaceutical combination of the present invention in either the form of its free base or any pharmaceutically acceptable salt thereof. Salts can be present alone or in mixture with free compound, e.g. the compound of the formula (I). Such salts of the compounds of formula (I) are formed, for example, as acid addition salts, preferably with organic or inorganic acids, from compounds of formula (I) with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it would be possible to use pharmaceutically unacceptable salts, for example picrates or perchlorates. For therapeutic use, only pharmaceutically acceptable salts or free compounds are employed (where applicable in the form of a medicament), as would be known to those skilled in the art. In view of the close relationship between the compound in free form and those in the form of its salts, any reference to the free compounds hereinbefore and hereinafter is to be understood as referring also to the corresponding pharmaceutically acceptable salts, as appropriate. Suitable counter-ions forming pharmaceutically acceptable salts are known in the field.

In the present invention, the pharmaceutical combination comprises an fibroblast growth factor receptor (FGFR) kinase inhibitor. The phrase “fibroblast growth factor receptor (FGFR) kinase inhibitor” as used herein refers to compounds which bind to one or more fibroblast growth factor receptors (e.g., FGFR1, FGFR2, FGFR3, FGFR4, or FGFR6) to modulate its function.

FGFR kinase inhibitors particularly useful in the present invention are those disclosed in WO 2006/000420, especially the compounds of formula (II) and salts, esters or N-oxides thereof, are a particular embodiment. WO2006/000420, which is hereby incorporated by reference in its entirety, discloses a group of compounds with high selectivity toward FGFRs

Especially preferred of the compounds of formula (II) is the specific compound 3-(2,6-dichloro-3,5-dimethoxy-phenyl)-1-{6-[4-(4-ethylpiperazin-1-l)-phenylamino]-pyrimidin-4-yl}-1-methyl-urea (herein referred to as “Compound B”) which has the formula:

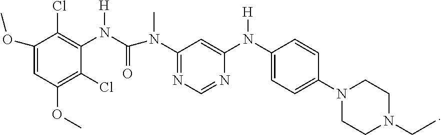 [SMILES: CCN1CCN(c2ccc(Nc3cc(N(C)C(=O)Nc4c(Cl)c(OC)cc(OC)c4Cl)ncn3)cc2)CC1]

Compound B and its preparation are described in Example 145 of WO2006/000420. This compound is highly selective for FGFR kinase receptors 1-4 (FGFR1, FGFR2, FGFR3, and FGFR4).

While this FGFR kinase inhibitor is of particular interest, also other FGFR kinase inhibitors are possible. Examples of other suitable FGFR kinase inhibitors include, but are not limited to, the following compounds (including pharmaceutically acceptable salts thereof):

TKI258 (previously known as CHIR258) is disclosed in WO02/22598 in example 109, as well as in Xin, X. et al., (2006), Clin. Cancer Res., Vol 12(16), p. 4908-4915; Trudel, S. et al., (2005), Blood, Vol. 105(7), p. 2941-2948), which are both hereby incorporated by reference in their entirety.

AZD-4547 (AstraZeneca) which has the formula:

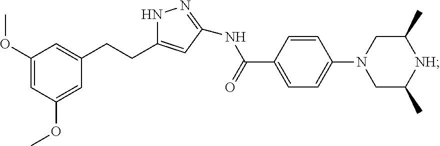

PD173074 (Imperial College London) (N-[2-[[4-(diethylamino)butyl]amino-6-(3,5-dimethoxyphenyl)pyrido[2,3-d]pyrimidin-7-yl]-N′-(1,1-dimethylethyl)urea which has the formula:

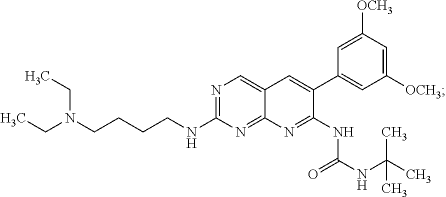 [SMILES: CCN(CC)CCCCNc1ncc2cc(-c3cc(OC)cc(OC)c3)c(NC(=O)NC(C)(C)C)nc2n1]

intedanib, dovitinib, brivanib (especially the alaninate), cediranib, masitinib, orantinib, ponatinib and E-7080 of the following formulae:

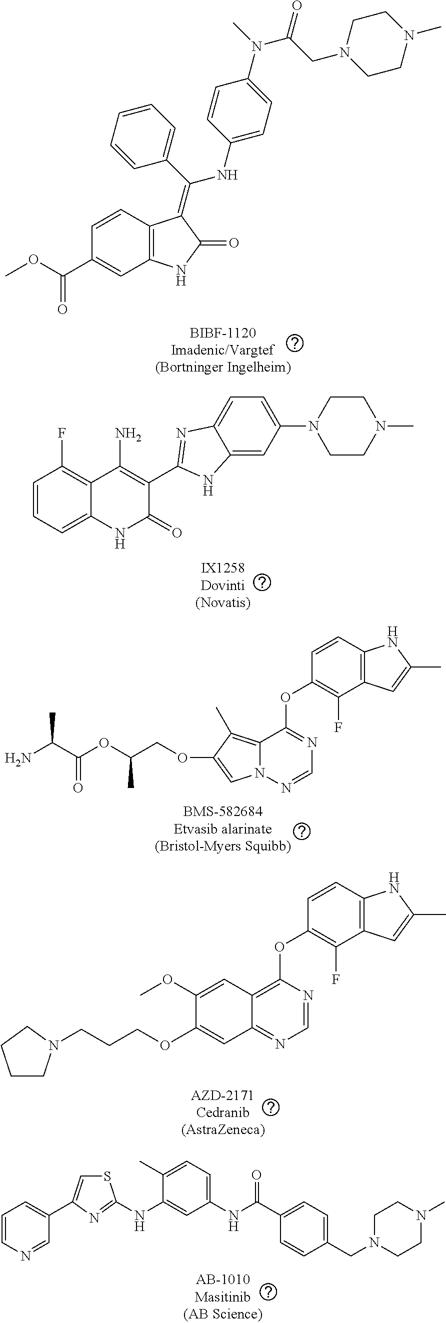

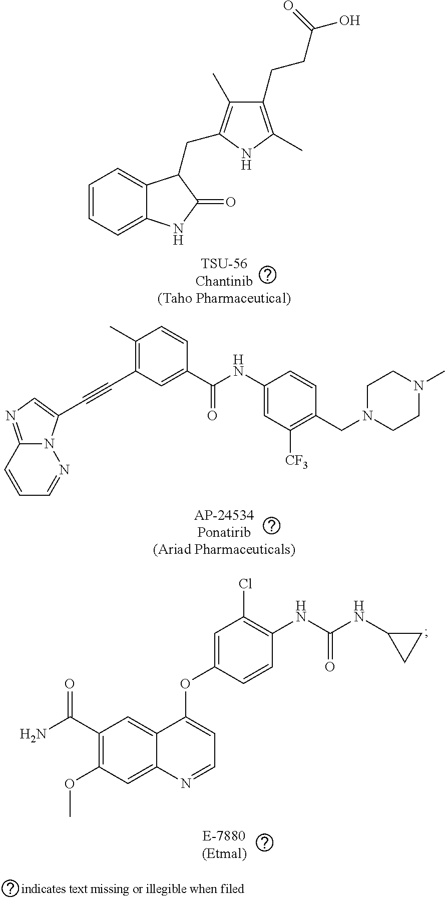 [SMILES: Cc1[nH]c(CC2C(=O)Nc3ccccc32)c(C)c1CCC(=O)O ; Cc1ccc(C(=O)Nc2ccc(CN3CCN(C)CC3)c(C(F)(F)F)c2)cc1C#Cc1cnc2cccnn12 ; COc1cc2nccc(Oc3ccc(NC(=O)NC4CC4)c(Cl)c3)c2cc1C(N)=O]

and the following antibodies or related molecules:
HGS1036/FP-1039 (Human Genome Science/Five Prime) (see also J. Clin. Oncol. 28:15s, 2010, which is hereby incorporated by reference): soluble fusion protein consisting of the extracellular domains of human FGFR1 linked to the Fc region of human Immunoglobulin G1 (IgG1), designed to sequester and bind multiple FGF ligands and lock activation of multiple FGF receptors; MFGR1877S (Genentech/Roche): monoclonal antibody; AV-370 (AVEO): humanized antibody; GP369/AV-396b (AVEO): FGFR-IIIb-specific antibody; and HuGAL-FR21 (Galaxy Biotech): monoclonal antibody (FGFR2).

The structure of the active agents identified by code nos., generic or trade names may be taken from the actual edition of the standard compendium “The Merck Index” or from databases, e.g., Patents International (e.g., IMS World Publications). The corresponding content thereof is hereby incorporated by reference.

Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the present invention can be prepared and administered as described in the cited documents, respectively. Also within the scope of this invention is the combination of more than two separate active ingredients as set forth above, i.e., a pharmaceutical combination within the scope of this invention could include three active ingredients or more.

In one embodiment, the FGFR kinase inhibitor used in the present invention is selected from the group consisting of TKI258, 3-(2,6-dichloro-3,5-dimethoxy-phenyl)-1-{6-[4-(4-ethylpiperazin-1-l)-phenylamino]-pyrimidin-4-yl}-1-methyl-urea, AZD4547, PD173074, intedanib, dovitinib, brivanib (especially the alaninate), cediranib, masitinib, orantinib, ponatinib, E-7080, HGS1036/FP-1039, MFGR1877S, GP369/AV-396b, and HuGAL-FR21 or a pharmaceutically acceptable salt thereof.

In a preferred embodiment, the FGFR kinase inhibitor used in the present invention is 3-(2,6-dichloro-3,5-dimethoxy-phenyl)-1-{6-[4-(4-ethylpiperazin-1-l)-phenylamino]-pyrimidin-4-yl}-1-methyl-urea or a pharmaceutically acceptable salt thereof.

In one embodiment, the present invention provides a pharmaceutical combination comprising (a) a compound of formula (I) or a pharmaceutically acceptable salt thereof; and (b) an FGFR kinase inhibitor.

In a further embodiment, the present invention provides a pharmaceutical combination comprising (a) a compound of formula (I) or a pharmaceutically acceptable salt thereof; and (b) an FGFR kinase inhibitor selected from the group consisting of TKI258, 3-(2,6-dichloro-3,5-dimethoxy-phenyl)-1-{6-[4-(4-ethylpiperazin-1-l)-phenylamino]-pyrimidin-4-yl}-1-methyl-urea, AZD4547, PD173074, intedanib, dovitinib, brivanib (especially the alaninate), cediranib, masitinib, orantinib, ponatinib, E-7080, HGS1036/FP-1039, MFGR1877S, GP369/AV-396b, and HuGAL-FR21 or a pharmaceutically acceptable salt thereof.

In a preferred embodiment, the present invention provides a pharmaceutical combination comprising (a) a compound of formula (I) or a pharmaceutically acceptable salt thereof; and (b) an FGFR kinase inhibitor 3-(2,6-dichloro-3,5-dimethoxy-phenyl)-1-{6-[4-(4-ethylpiperazin-1-l)-phenylamino]-pyrimidin-4-yl}-1-methyl-urea or a pharmaceutically acceptable salt thereof.

It has been surprisingly found that the combination of the alpha-isoform specific phosphatidylinositol 3-kinase inhibitor compound of formula (I) and an FGFR kinase inhibitor, especially 3-(2,6-dichloro-3,5-dimethoxy-phenyl)-1-{6-[4-(4-ethylpiperazin-1-1)-phenylamino]-pyrimidin-4-yl}-1-methyl-urea, possesses significant beneficial therapeutic properties, e.g., synergistic interaction or strong anti-proliferative activity, which render it particularly useful for the treatment of cancer.

Suitable cancers that can be treated with the combination of the present invention include, but are not limited to sarcoma, lymphomas, cancer of the lung, bronchus, prostate, breast (including sporadic breast cancers and sufferers of Cowden disease), pancreas, gastrointestine, colon, rectum, colon, colorectal adenoma, thyroid, liver, intrahepatic bile duct, hepatocellular, adrenal gland, stomach, gastric, glioma, glioblastoma, endometrial, melanoma, kidney, renal pelvis, bladder, uterine corpus, cervix, vagina, ovary, multiple myeloma, esophagus, adrenal gland, pituitary, a leukemia, acute myelogenous leukemia, chronic myelogenous leukemia, lymphocytic leukemia, myeloid leukemia, brain, oral cavity, pharynx, larynx, small intestine, non-Hodgkin lymphoma, melanoma, villous colon adenoma, a neoplasia, a neoplasia of epithelial character, a mammary carcinoma, basal cell carcinoma, squamous cell carcinoma, actinic keratosis, a tumor of the neck or head, polycythemia vera, essential thrombocythemia, myelofibrosis with myeloid metaplasia, Waldenstroem disease, 8p11 myeloproliferative syndrome (EMS), retinoblastoma, synovial sarcoma, hematological malignancies, benign lichenoid keratosis, solar lentigo, beborrhoeic keratosis, epidermal nevi or any combination thereof. It is understood that the cancer may be benign or metastatic.

Suitable cancers that can be treated with the combination of the present invention may be a solid tumor or liquid tumor. In one embodiment, the cancer treated with the combination of the present invention is a solid tumor. The term “solid tumor” especially means cancer of the breast, ovary, colon, gastrointestine, cervix, lung (including small cell lung cancer and non-small cell lung cancer), head and neck, urinary bladder, or prostate. The term “liquid tumor” especially means leukemias (e.g., acute myelogenous leukemia, chronic myelogenous leukemia, lymphocytic leukemia, myeloid leukemia), lymphomas (e.g., Non-Hodgkin lymphoma), multiple myeloma, and hematological malignancies.

In a preferred embodiment, the cancer treated with the combination of the present invention is selected from the group consisting of breast cancer, bladder cancer, endometrial cancer, and ovarian cancer.

The pharmaceutical combination of the present invention may be particularly useful for the treatment of various cancers that are mediated by, especially dependent on, the activity of PI3K (particularly the alpha-subunit of PI3K) and/or FGFR kinase, respectively. Such cancers mediated by the activity of PI3K may include, but are not limited to, those showing amplification of PI3K alpha, somatic mutation of PIK3CA or mutations and translocation of p85α that serve to up-regulate the p85-p110 complex. Such cancers mediated by the activity of FGFR kinase may include, but are not limited to, gene amplification of FGFR1, FGFR2, FGFR3 or FGFR4; translocation and fusion of FGFR1 to other genes; or somatic activating mutations of FGFR1, FGFR2, FGFR3 or FGFR4. In one embodiment, the cancer treated with the combination of the present invention is a cancer having (a) amplification of PI3K alpha or somatic mutation of PIK3CA, and (b) gene amplification or somatic activating mutations of FGFR1, FGFR2, FGFR3 or FGFR4. Preferably, the cancer treated with the combination of the present invention is a breast cancer having (a) amplification of PI3K alpha or somatic mutation of PIK3CA, and (b) gene amplification or somatic activating mutations of FGFR1, FGFR2, FGFR3 or FGFR4.

Thus in one aspect, the present invention provides the use of PIK3CA amplification or somatic activating mutation and FGFR1, FGFR2, FGFR3 or FGFR4 amplification or somatic activating mutations as biomarkers to select patients who are likely to respond to the treatment of the pharmaceutical combination of the invention. Many somatic activating mutations of those genes leading to malignancy have been identified and well known to a skilled person. Gene amplification can be detected by fluorescence in site hybridization (FISH) or qRT-PCR, using healthy sample as negative control. In one embodiment, the present invention provides a pharmaceutical combination comprising (a) a compound of formula (I) or a pharmaceutically acceptable salt thereof and (b) an FGFR kinase inhibitor for simultaneous, separate or sequential use in the treatment of a cancer.

In a further embodiment, the present invention provides a pharmaceutical combination comprising (a) a compound of formula (I) or a pharmaceutically acceptable salt thereof and (b) an FGFR kinase inhibitor, for simultaneous, separate or sequential use in the treatment of a solid tumor.

In a further embodiment, the present invention provides a pharmaceutical combination comprising (a) a compound of formula (I) or a pharmaceutically acceptable salt thereof and (b) an FGFR kinase inhibitor, for simultaneous, separate or sequential use in the treatment of a liquid tumor.

In a further embodiment, the present invention provides a pharmaceutical combination comprising (a) a compound of formula (I) or a pharmaceutically acceptable salt thereof and (b) an FGFR kinase inhibitor, for simultaneous, separate or sequential use in the treatment of a cancer selected from the group consisting of breast cancer, bladder cancer, endometrial cancer, and ovarian cancer.

In a further embodiment, the present invention provides a pharmaceutical combination comprising (a) a compound of formula (I) or a pharmaceutically acceptable salt thereof and (b) an FGFR kinase inhibitor, for simultaneous, separate or sequential use in the treatment of a cancer having (a) amplification of PI3K alpha or somatic mutation of PIK3CA, and (b) gene amplification or somatic activating mutations of FGFR1, FGFR2, FGFR3 or FGFR4. Preferably, said cancer is breast cancer.

To demonstrate that the pharmaceutical combination of the present invention is particularly suitable for the effective treatment of a cancer with good therapeutic margin and other advantages, the preclinical experiment(s) described in Examples 1, 2 or 3 herein can be carried out by a person skilled in the art. Established correlations between tumor models and effects seen in man suggest that synergy in animals may, e.g., be demonstrated in the RT112 urinary bladder transitional cell carcinoma model, MFE280 endometrial cancer model and/or AN3CA endometrial cancer model as described in the Examples below.

Alternatively, clinical trials can be carried out in a manner known to the skilled person. Suitable clinical studies are, e.g., open label, dose escalation studies in patients with cancers. Such studies can be used to demonstrate in particular the synergism of the active ingredients of the combination of the invention. The beneficial effects can be determined directly through the results of these studies which are known as such to a person skilled in the art. Such studies are, in particular, suitable to compare the effects of a monotherapy using the active ingredients and a combination of the invention. Preferably, the dose of active ingredient (a) is escalated until the Maximum Tolerated Dosage is reached, and active ingredient (b) is administered with a fixed dose. Alternatively, the active ingredient (a) is administered in a fixed dose and the dose of active ingredient (b) is escalated. Each patient receives doses of the active ingredient (a) either daily or intermittent. The efficacy of the treatment can be determined in such studies, e.g., after 12, 18 or 24 weeks by evaluation of symptom scores every 6 weeks.

The active ingredients of the pharmaceutical combination of the present invention are preferably formulated or used to be jointly therapeutically effective. This means in particularly that there is at least one beneficial effect, e.g., a mutual enhancing of the event of the active ingredient (a) and (b), in particularly synergism, e.g. a more than additive effect, e.g., additional advantageous effects (such as a further therapeutic effect not found for any of the single compounds), less side effects, a combined therapeutic effect in a non-effective dosage of one or both of the active ingredients (a) and (b), and very preferably a clear synergism of the active ingredients (a) and (b).

The administration of a pharmaceutical combination of the invention is expected to result not only in a beneficial effect, e.g., a synergistic therapeutic effect, e.g., with regard to alleviating, delaying progression of or inhibiting the symptoms, but also in further surprising beneficial effects, e.g., fewer side effects, an improved quality of life or a decreased morbidity, compared with a monotherapy applying only one of active ingredient (a) or active ingredient (b) used in the combination of the invention.

A further expected benefit is that lower doses of the active ingredients of the pharmaceutical combination of the invention can be used, e.g., that the dosages need not only often be smaller but are also applied less frequently, which may diminish the incidence or severity of side effects. This is in accordance with the desires and requirements of the patients to be treated.

In one embodiment, the present invention provides the use of a pharmaceutical combination comprising a compound of formula (I) or a pharmaceutically acceptable salt thereof and an FGFR kinase inhibitor for the preparation of a medicament for the treatment of a cancer.

In one embodiment, the present invention provides the use of a compound of formula (I) or a pharmaceutically acceptable salt thereof for the preparation of a medicament for use in combination with an FGFR inhibitor for the treatment of cancer.

In one embodiment, the present invention provides the use of a pharmaceutical combination comprising a compound of formula (I) or a pharmaceutically acceptable salt thereof and an FGFR kinase inhibitor for the treatment of a cancer.

In one embodiment, the present invention provides a method of treating a cancer comprising administering to a subject, especially a human, having said cancer a jointly therapeutically effective amount of a compound of formula (I) or a pharmaceutically acceptable salt thereof and an FGFR kinase inhibitor. Such compound of formula (I) and FGFR kinase inhibitor may be administered either as a single pharmaceutical composition, or as separate pharmaceutical compositions administered simultaneously, separately or sequentially.

In a further embodiment, the present invention provides a method of treating a cancer comprising administering to a subject, especially a human, having said cancer a therapeutically effective amount of a compound of formula (I) or a pharmaceutically acceptable salt thereof and a therapeutically effective amount of FGFR kinase inhibitor.

Pharmaceutical compositions according to the present invention can be prepared in a manner known per se and are those suitable for enteral, such as oral or rectal, and parenteral administration to subjects, especially humans, comprising a therapeutically effective amount of at least one active agent alone or in combination with one or more pharmaceutically acceptable carrier. In one embodiment of the invention, one or more of the active ingredients are administered orally.

The active ingredients of the pharmaceutical combination of the present invention may be administered together in a single pharmaceutical composition, separately into two or more unit dosage forms, or sequentially. The combination of the compound of formula (I) and an FGFR kinase inhibitor can administered as a kit of parts that can be dosed independently or by use of different fixed combinations with distinguished amounts of the active ingredients, i.e., simultaneously or at different time points.

The pharmaceutical compositions for separate administration of active ingredient (a) and active ingredient (b) or for the administration in a fixed combination (i.e., a single pharmaceutical composition or medicament comprising at least two active ingredients (a) and (b)) according to the invention may be prepared in a manner known per se and are those suitable for enteral (such as oral or rectal), topical, and parenteral administration to subjects, preferably humans. Such pharmaceutical compositions comprise a therapeutically effective amount of at least one active ingredient alone, as indicated above, or in combination with one or more pharmaceutically acceptable carriers.

Pharmaceutical compositions may comprise one or more pharmaceutical acceptable carriers and may be manufactured in conventional manner by mixing one or both combination partners with one or more pharmaceutically acceptable carriers. Examples of pharmaceutically acceptable diluents include, but are not limited to, lactose, dextrose, mannitol, and/or glycerol, and/or lubricants and/or polyethylene glycol. Examples of pharmaceutically acceptable acceptable binders include, but are not limited to, magnesium aluminum silicate, starches, such as corn, wheat or rice starch, gelatin, methylcellulose, sodium carboxymethylcellulose and/or polyvinylpyrrolidone, and, if desired, pharmaceutically acceptable disintegrants include, but are not limited to, starches, agar, alginic acid or a salt thereof, such as sodium alginate, and/or effervescent mixtures, or adsorbents. Pharmaceutical compositions may also include dyes, flavorings and sweeteners. It is also possible to use the compounds of the present invention in the form of parenterally administrable compositions or in the form of infusion solutions. The pharmaceutical compositions may be sterilized and/or may comprise excipients, for example preservatives, stabilizers, wetting compounds and/or emulsifiers, solubilisers, salts for regulating the osmotic pressure and/or buffers.

In particular, a therapeutically effective amount of each active ingredient of the pharmaceutical combination of the invention may be administered simultaneously or sequentially and in any order, and the components may be administered separately or as a fixed combination. For example, the method of treating a cancer according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual active ingredients of the combination of the invention may be administered separately at different times during the course of therapy or concurrently in divided or single combination forms. Furthermore, the term “administering” also encompasses the use of a pro-drug of an active ingredient that convert in vivo to the active ingredient as such. The instant invention is therefore to be understood as embracing all such regimens of simultaneous or alternating treatment and the term “administering” is to be interpreted accordingly.

The effective dosage of each of active ingredient (a) or active ingredient (b) employed in the pharmaceutical combination of the invention may vary depending on the particular compound or pharmaceutical composition employed, the mode of administration, the condition being treated, the severity of the condition being treated. Thus, the dosage regimen of the combination of the invention is selected in accordance with a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the active ingredient required to prevent, counter or arrest the progress of the condition. Optimal precision in achieving concentration of active ingredient within the range that yields efficacy requires a regimen based on the kinetics of the active ingredient's availability to target sites. This involves a consideration of the distribution, equilibrium, and elimination of an active ingredient.

For purposes of the present invention, a therapeutically effective dose will generally be a total daily dose administered to a host in single or divided doses.

The compound of formula (I) may be administered to a host in a daily dosage range of, for example, from about 0.05 to about 50 mg/kg body weight of the recipient, preferably about 0.1-25 mg/kg body weight of the recipient, more preferably from about 0.5 to 10 mg/kg body weight of the recipient. For administration to a 70 kg person, the dosage range of the compound of formula (I) would most preferably be about 35-700 mg daily.

The compound 3-(2,6-dichloro-3,5-dimethoxy-phenyl)-1-{6-[4-(4-ethylpiperazin-1-l)-phenylamino]-pyrimidin-4-yl}-1-methyl-urea may be administered to a host in a daily dosage range of, for example, from about 0.03 to 2.5 mg/kg per body weight. An indicated daily dosage in the larger warm-blooded animal, e.g. humans, is in the range from about 0.5 mg to about 100 mg, conveniently administered, e.g. in divided doses up to four times a day or in retard form. Suitable unit dosage forms for oral administration comprise from about 1 to 50 mg active ingredient.

In one embodiment, the present invention provides a pharmaceutical composition comprising a compound of formula (I) or a pharmaceutically acceptable salt thereof and an FGFR kinase inhibitor, preferably 3-(2,6-dichloro-3,5-dimethoxy-phenyl)-1-{6-[4-(4-ethylpiperazin-1-l)-phenylamino]-pyrimidin-4-yl}-1-methyl-urea or a pharmaceutically acceptable salt thereof. Such pharmaceutical composition may be simultaneously, separately, or sequentially use in the treatment of a cancer.

In one embodiment, the present invention provides a pharmaceutical composition comprising a compound of formula (I) or a pharmaceutically acceptable salt thereof and an FGFR kinase inhibitor, preferably 3-(2,6-dichloro-3,5-dimethoxy-phenyl)-1-{6-[4-(4-ethylpiperazin-1-l)-phenylamino]-pyrimidin-4-yl}-1-methyl-urea or a pharmaceutically acceptable salt thereof, for simultaneously, separately, or sequentially use in the treatment of a cancer mediated by the activity of PI3K (particularly the alpha-subunit of PI3K) and/or FGFR kinase.

In one embodiment, the present invention further relates to a kit comprising a compound of formula (I) according to claim 1 or a pharmaceutically acceptable salt thereof, and a package insert or label providing instruction for treating a cancer by co-administering at least one FGFR kinase inhibitor.

In one embodiment, the present invention further relates to a kit comprising a compound of formula (I) or a pharmaceutically acceptable salt thereof, and an FGFR kinase inhibitor, and a package insert or other labeling including directions for treating a cancer. The following Examples illustrate the invention described above; they are not, however, intended to limit the scope of the invention in any way. The beneficial effects of the pharmaceutical combination of the present invention can also be determined by other test models known as such to the person skilled in the pertinent art.

### Example 1

Potential synergistic interactions between Compound A and Compound B are assessed in RT112 urinary bladder transitional cell carcinoma cells, MFE280 endometrial cancer cells and AN3CA endometrial cancer cells.

### Material and Methods

Compound preparation: Each of Compound A and Compound B are dissolved in DMSO as a 10 mM stock. Serial dilutions, as indicated below, are made in culture medium before adding to the cell cultures.

Cell lines: The human endometrium adenocarcinoma cell line AN3CA is obtained from ATCC (HTB-111). The human endometrium adenocarcinoma cell line MFE280 is obtained from Deutsche Sammlung von Mikroorganismen und Zellkulturen GmbH (Braunschweig, Germany, ACC-410). The human urinary bladder transitional cell carcinoma RT112 cell line is obtained from Deutsche Sammlung von Mikroorganismen und Zellkulturen GmbH (Braunschweig, Germany, ACC-418).

AN3CA cells are maintained in Eagle's Minimum Essential Medium (EMEM) (Amimed, catalog no. 1-31S01-I) supplemented with 10% FCS, 2 mM L-glutamine (Amimed, catalog no. 5-10K00-H), 1 mM sodium pyruvate (AMIMED#5-60-F00-H), and 100 units/ml Penicillin/Streptomycin (Amimed, catalog no. 4-01F00-H). MFE280 are maintained in 40% RPMI 1640 (Amimed, catalog no. 1-41F01) plus 40% Minimum Essential Medium (MEM) (Amimed, catalog no. 1-31F01-I) supplemented with 20% fetal calf serum (FCS), 2 mM L-glutamine (Amimed, catalog no. 5-10K00-H) and insulin-transferin-sodium selenite (Invitrogen, catalog no. 51500-056) and 100 units/ml Penicillin/Streptomycin (Amimed, catalog no. 4-01F00-H). RT112 cells are grown in Minimum Essential Medium with Earle's balanced salts (MEM-EBS) (Amimed, catalog no. 1-31F0-I) supplemented with 1% L-glutamine (Amimed, catalog no. 5-10K00-H), 1% Minimum Essential Medium with Non-Essential Amino Acids (MEM NEAA) (Amimed, catalog no. 5-13K00-H), 1% sodium pyruvate (Amimed, catalog no. 5-60F00-H) and 10% FCS (Gibco, catalog no. 10082-147).

Proliferation experiments: Cellular proliferation assays are performed in 384-well plate format. AN3CA cells are diluted in medium to a density of 2.22×10⁴ cells/ml and are seeded in 45 μl per well (yielding 1000 cells/well) to 384-well microtiter plates (Corning #3701) using a μFill liquid dispenser (BIOTEK). Likewise MFE280 cells are diluted in medium to a density of 4.44×10⁴ cells/ml and seeded in 45 μl per well (yielding 2000 cells/well). RT112 cells are diluted in medium to a density of 2.5×10⁴ cells/ml and seeded in 20 μL per well (yielding 500 cells/well) to 384-well microtiter plates (Corning #3683) using a μFill liquid dispenser (BIOTEK).

After 24 hours incubation at 37° C. in a 5-7.5% CO₂ humidified atmosphere, compounds are added to the cells. Cells are incubated at 37° C. in a 5-7.5% CO₂ humidified atmosphere for a further 5 days.

For the AN3CA, MFE280 cells, the effect of the compounds on cell viability is assessed by the addition of resazurin as previously described (Hamid et al. 2004). Five microliters of Resazurin solution (130 ug/ml PBS; SIGMA # R7017-5G) is added per well and the plates incubated for 4-6 hours at 37° C. and 5% CO₂. Thereafter, fluorescence is measured using a microplate reader with the following settings: AN3-CA and MFE-280 (SynergyHT, excitation 560/15 nm and Emission 590/20 nm).

For the RT112 cells, the effect of the compounds on cell viability is assessed by quantification of cellular ATP using the CellTiter-Glo kit (Promega, Cat#G7571) as per supplier instructions. Briefly, 25:1 reconstituted CellTiter-Glo reagent is added to 25 μl cell culture volume in the assay plate and is mixed for 3 minutes on an orbital shaker to aid cell lyses. The plate is then incubated at room temperature for 10 minutes before luminescence was recorded on a M1000 multipurpose micro plate reader (TECAN).

Drug combinations: For AN3CA, MFE280 cells, serial 1:2.5 dilutions of compound A and compound B are prepared in DMSO using a Velocity Bravo (Agilent) robotic liquid handler. For AN3CA and MFE280 cells, drug combinations are prepared by further dilution in medium using a FreedomEVO (TECAN) robotic liquid handler, combining 2 μl individual compound A dilutions and 2 μl individual compound B dilutions and 196 μl medium of which 5 μl were added to the 45 μl cell culture volume in the assay plate. Serial dilutions of single agents compound A and compound B are processed accordingly. Assays are performed in this format allow for a 14×14 checkerboard dose matrix including single agent titration and DMSO control. Assays are done using three replicate plates.

For RT112 cells, serial 1:3 dilutions of compound A and compound B are prepared in DMSO using a FreedomEVO (TECAN) robotic liquid handler and combined at 1′000-fold desired final assay concentration in a 384-well compound master plate. Drug combinations are prepared by further dilutions in medium using a FreedomEVO (TECAN) robotic liquid handler, combining 1 μl master dilution and 199 μl medium, of which 5 μl are added to the 25 μl cell culture volume in the assay plate. Corresponding serial dilutions of single agents compound A and compound B are added to the cells. Assays performed in this format allow for a six extended 5×5 checkerboard dose matrices per plate, including single agent titration and DMSO control. Highest and lowest concentrations of each compound are combined with each other only. Assays were performed using two replicate plates.

Calculation of combination effects: The synergistic effects of drug combination are determined relative to a null expectation that is calculated from the single agent activities based on standard non-interaction models (Berenbaum, M. C., Pharmacol. Rev. 41, 93-141 (1989)). Several such models are in use, the most common being Loewe dose additivity (Loewe, S., Die quantitativen Probleme der Pharmakologie. Ergeb. Physiol. 27: 47-187 (1928), hereby incorporated by reference)), which is the expected response if both agents inhibit the same molecular target by means of the same mechanism.

The factorial dose matrix is used to sample all mixtures of the two serially diluted single agents. Growth inhibition measurements relative to vehicle-treated samples are determined and visualized over the matrix using a color scale. To determine synergy, each measurement is compared to expected values derived from the single-agent data along the left and bottom edges of the matrix. Synergy is also described using an isobologram, which compares the doses needed to reach 50% inhibition along an equal-effect contour to those along a predicted contour based on a model of dose additivity.

Dose shifting relative to Loewe additivity is measured using a combination index (Chou and Talalay 1984). CI=CX/ICX+CY/ICY, which measures the fractional shift between the most potent combination doses (CX and CY) and the single agents' 50% inhibitory concentrations (IC50(X) and IC50(Y)). Combination indices (CI) of >1.1 are indicative of antagonism, CI of <1.1 to >0.9 are indicative of nearly additive activity and CI of <0.9 are indicative of synergism.

In these experiments using dose matrices, the synergy is measured using a synergy score S=In fX In fY Σdoses max (0, Zdata) (Zdata-ZLoewe), between measured effects Zdata and a Loewe additive surface ZLoewe derived from the single-agent curves. This synergy score is a positive gated, effect-weighted volume over Loewe additivity, adjusted for variable dilution factors fX,fY. A more detailed explanation of the technique and calculation can be found in Lehar et al., Nat. Biotechnol. 27(7): 659-666 (July 2009), which is hereby incorporated by reference.

### Results:

FIGS. 1, 2 and 3 describe the synergistic effect between Compound A and Compound B observed in RT112 urinary bladder carcinoma cells, MFE280 endometrial cancer cells and AN3CA endometrial cancer cells respectively.

FIG. 1: growth inhibition experiments are performed with RT112 using concentration ranges between 270 and 0.37 nM for compound B, and between 11′000 and 15 nM for compound A. Factorial dose response displayed in the Dose Matrix plot shows the extent of growth inhibition by the two agents alone and in combination, and the Loewe Excess plot demonstrates the existence of multiple drug concentrations at which the two drugs interact in a supra-additive manner. The resulting isobologram calculated for 50% growth inhibition indicates synergism for most drug combinations. The best CI at 50% is calculated as 0.57±0.08, indicating moderate synergy. The synergy score is determined as 3.94 (see method section).

FIG. 2: growth inhibition experiments are performed with MFE280 using concentration ranges of compound A and compound B between 10′000 and 1 nM. Factorial dose response is displayed in the Dose Matrix plot shows the extent of growth inhibition by the two agents alone and in combination, and the Loewe Excess plot demonstrates the existence of multiple drug concentrations at which the two drugs interact in a supra-additive manner (10′000 to 260 nM compound A plus 10′000 to 0.17 nM compound B), and only a few sub-additive drug combinations (negative values, 6.6 to 0.2 nM compound A plus 6.6 nM compound B). The resulting isobologram is calculated for 50% growth inhibition indicates substantial synergism for most drug combinations. The best CI at 50% is calculated as 0.05±0.08, indicating strong synergy. The synergy score is determined as 16.

FIG. 3: growth inhibition experiments are performed with AN3CA using concentration ranges of compound A and compound B between 10′000 and 1 nM. Factorial dose response shown in Dose Matrix indicates dose-dependent growth inhibition and the Loewe Excess plot shows wide distribution of synergistic drug combinations (10′000 to 0.2 nM compound A plus 100 to 0.17 nM compound B) and a smaller area of sub-additive drug combinations (negative values, 260 to 0.2 nM compound A plus 1′600 to 640 nM compound B). The resulting isobologram calculated for 50% growth inhibition indicates substantial synergism for most drug combinations. The best CI at 50% is calculated as 0.12±0.02, indicating strong synergy. The synergy score is determined as 9.43.

Thus, it has been demonstrated that Compound A and Compound B have synergistic effects in inhibiting proliferation of various cancer cell lines.

### Example 2

The following experiment demonstrates the efficacy and anti-proliferative activity of the alpha-isoform specific phosphatidylinositol 3-kinase inhibitor Compound A in combination with FGFR kinase inhibitor Compound B in the treatment of urinary bladder carcinoma:

### Materials and Methods:

Animals: Experiments are performed in female Hsd: Athymic Nude-nu mice obtained from Harlan Cpb, Germany. Animals are from 8 to 14 weeks of age at treatment start and are housed under Optimized Hygienic Conditions in Makrolon type III cages (max. 10 animals per cage) with free access to food and water

Establishment of RT112 tumors: The RT112 human urinary bladder carcinoma cell line was initially derived from a female patient with untreated primary urinary bladder carcinoma in 1973. RT112 cells are cultured as in Example 1. To establish tumors, RT112 cells suspended in Hanks' balanced salt solution (HBSS) containing 50% Matrigel (BD #356234) are injected subcutaneously into the right flank. A total of 5×10⁶ cells in 100 μl are injected per animal.

Evaluation of antitumor activity: Treatments are initiated when the mean tumor volumes were approximately 180 mm³ (14 days post tumor cells injection). Body weights and tumor volumes are recorded twice a week. Tumor volumes are measured with calipers and determined according to the formula length×diameter²×π/6. In addition to presenting changes of tumor volumes over the course of treatments, antitumor activity is expressed as T/C % (mean change of tumor volume of treated animals/mean change of tumor volume of control animals)×100. Regressions (%) are calculated according to the formula ((mean tumor volume at end of treatment-mean tumor volume at start of treatment)/mean tumor volume at start of treatment)×100.

Preparation of Compound Solutions: Vehicle control is prepared by mixing 250 μl 1-methyl-2-pyrrolidone (“NMP”)(Fluka #69118), 750 μl Polyethylene glycol 300 (“PEG300”)(Fluka, #81162), 500 μl Solutol HS15 (BASF, #51633963) and 1000 μl water.

Solutions of Compound A are prepared as follows: (a) For single agent application, 12.5 mg Compound A is dissolved in 250 μl NMP, 750 μl PEG300, 500 μl Solutol (HS15, BASF), and 1000 μl water, (b) For combination application, 12.5 mg Compound A is dissolved in 125 μl NMP, 375 μl PEG300, 250 μl solutol (HS15, BASF), and 500 μl water.

Solutions of Compound B are prepared as follows: (a) For single agent application, 3 mg Compound B is dissolved in 2000 μl PEG300 and 1000 μl Glucose 5% (B. Braun Medical AG, catalog no. 29550), (b) For combination application, 3 mg Compound B is dissolved in 1000 μl PEG300 and 500 μl Glucose 5% (B. Braun Medical AG, catalog no. 29550).

Compound A and Compound B are sonicated in a water bath (33 kHz).

Statistics: For all tests, the level of significance is set at p<0.05. For tumor volumes, comparisons between treatment groups and vehicle control group are done using one-way ANOVA followed by Dunnett's test. The level of significance of body weight change within a group between the start and the end of the treatment period is determined using a paired t-test. Comparisons of delta body weighs between treatment and vehicle control groups are performed by a one-way ANOVA followed by a post hoc Dunnett's test. Calculations are performed using GraphPad Prism 6 for Windows (GraphPad Software Inc.).

Estimation of drug-drug interaction: an approximation of drug interactions is made using the method described by Clarke R., “Issues in experimental design and endpoint analysis in the study of experimental cytotoxic agents in vivo in breast cancer and other models”, Breast Cancer Res. Treat., 46, 255-78 (1997) (“Clark 1997”). This is applied to ΔTV (Mean tumor volume at the end of the treatment period—mean tumor volume at the beginning of the treatment period).

### Results:

Upon performance of the foregoing experiment, the results are summarized in the table below: Ten millions RT-112 cells are injected subcutaneously into the right flank of female athymic nude mice on Day 0. On day 14, tumors are measured and mice are randomized into treatment groups with 8 mice per group. Mice are treated with vehicle, 50 mg/kg Compound A (orally every day), 10 mg/kg Compound B (orally every day) or the combination of the two agents for 14 consecutive days. Statistics on Δ tumor volumes are performed with a one-way ANOVA, post hoc Dunnett's (*p<0.05 vs. vehicle controls) to compare treatment groups against the vehicle control group. For body weights, paired t tests between the weights at the beginning and the end of the treatment period are performed (*p<0.05).

| | Tumor response | | | Host response | | |
|---|---|---|---|---|---|---|
| | | | Mean change of | Mean change of | % change | Survival |
| | T/C | Regression | tumor volume | body weight | of body | (survivors/ |
| Treatment | (%) | (%) | (mm³ ± SEM) | (g ± SEM) | weight | total) |
| Vehicle | 100 | — | 267.7 ± 67.5 | 0.7 ± 0.7 | 2.5 | 8/8 |
| 10 ml/kg, p.o., q24 h | | | | | | |
| Compound B 10 mg/kg, | 54.4 | — | 145.6 ± 66.5 | 2.1* ± 0.6 | 8.2 | 8/8 |
| p.o., q24 h | | | | p < 0.05 | | |
| Compound A 50 mg/kg, | — | −1.1 | −2.0* ± 24.4 | −0.2 ± 0.4 | −0.8 | 8/8 |
| p.o., q24 h | | | p < 0.05 | | | |
| Combination | — | −62.4 | −113.2* ± 15.2 | −1.5 ± 0.7 | −5.8 | 8/8 |
| Compound B (10 mg/kg, | | | p < 0.05 | | | |
| p.o., q24 h) and | | | | | | |
| Compound A (50 mg/kg, | | | | | | |
| p.o., q24 h) | | | | | | |

FIG. 4: the graph shows the tumor volume changes over the course of treatment. Compound A given as a single agent produces statistically significant tumor regression of 1.1% when compared to vehicle controls (p<0.05, ANOVA post hoc Dunnett's). Compound B given as a single agent produces a non-statistically significant tumor growth inhibition of 54.4% compared to vehicle controls. Compound A and Compound B when administered together produce a statistically significant tumor regression of 62.4%.

FIG. 5: the graph shows the body weight changes over the course of treatment. The body weight change during the treatment period was statistically significant only in the group treated with Compound B (+8.2%, p<0.05, paired t test). Moreover, the body weight changes in the vehicle group was significantly different from the body weight changes in the combination chemotherapy group (p<0.05, one way ANOVA, post hoc Dunnett's).

A compound interaction analysis with the method described by Clarke 1997 is conducted. In accordance with this method, the experimental data is interpreted as follows: antagonism is predicted when the calculation AB/C>B/C×A/C, additive interaction is predicted when the calculation AB/C=B/C×A/C, and synergistic when AB/C<B/C×A/C. For this experiment, the experimental data clearly demonstrates a synergistic interaction for the combination as shown in the following table:

| | Vehicle | Cmpd B | Cmpd A | | | | | | | |
|---|---|---|---|---|---|---|---|---|---|---|
| | Control | only | only | Combo | | | | | | |
| | (C) | (B) | (A) | (AB) | B/C | A/C | B/C × A/C | AB/C | Difference | Result |
| Final | 449.2 | 327.2 | 179.4 | 68.3 | 0.728 | 0.399 | 0.291 | 0.152 | −0.139 | Synergy |
| Tumor | | | | | | | | | | |
| Volume | | | | | | | | | | |
| Mean change | 267.7 | 145.6 | −2 | −113.2 | 0.544 | −0.007 | −0.004 | −0.423 | −0.42 | Synergy |
| of tumor | | | | | | | | | | |
| volume | | | | | | | | | | |

### Example 3

The following experimental demonstrates the effect on inhibition of downstream signaling by the alpha-isoform specific phosphatidylinositol 3-kinase inhibitor Compound A in combination with FGFR kinase inhibitor Compound B in the treatment of urinary bladder carcinoma.

### Materials and Methods:

Animals: Experiments are performed in female Hsd: Athymic Nude-nu mice obtained from Harlan Cpb, Germany. Animals are from 8 to 14 weeks of age at treatment start and housed under Optimized Hygienic Conditions in Makrolon type III cages (max. 10 animals per cage) with free access to food and water

Establishment of RT112 tumors and compound treatments: The RT112 human urinary bladder carcinoma cell line was initially derived from a female patient with untreated primary urinary bladder carcinoma in 1973 (Marshall et al. 1977, Masters et al. 1986). RT112 cells are cultured as in Example 1. Tumor establishment is done as in Example 2. Treatments are initiated when the mean tumor volumes are approximately 240 mm³ (20 days post tumor cells injection). Tumor-bearing mice are treated for 7 consecutive days with compound A and compound B at doses 50 mg/kg and 10 mg/kg, respectively, as single agents or in combination.

Preparation of Compound Solutions: Vehicle control, compound A and compound B are prepared as in Example 2.

Dissection: 2 hours after the last treatment animals are anaesthetized by Forene inhalation narcosis and euthanized by cervical dislocation. Each tumor is dissected, is cut into two equal pieces and is processed for formalin-fixed paraffin embedded sectioning. For that purpose, tumors are fixed immediately after dissection in a 10% neutral buffered formalin solution for exactly 24 hours at room temperature. After fixation, dissected tumors are rinsed in PBS and processed for dehydration, clearing and paraffinisation under vacuum conditions in the TPCduo apparatus (Medite, Switzerland) according to the manufacturer's instructions. Tumors are then embedded in paraffin, and 3 μm sections prepared, mounted on polylysine-coated microscope slides and dried at 37° C. for 16 h.

Immunohistochemistry: The various primary antibodies used for immunohistochemistry are described in the below table:

| Antibodies | Species | Clone | References | Dilution range |
|---|---|---|---|---|
| AKTpS473 | rmAb | D9E | CST, cat.4060 | 1/50 |
| MAPK(Erk1, 2) | rmAb | D13.14.4E | CST, cat.4370 | 1/200 |
| p44_42 | | | | |

Slides are mounted on a Ventana Discovery XT immunostainer and are processed for automated IHC by using a ChromoMAP kit and an OmniMAP anti Rabbit polymer-based amplification system. Briefly, slides are dewaxed, hydrated and antigen retrieval is done by incubating slides with CCultra solution at 100° C. for 44 min for pAKT or for 36 min for pMAPK. Endogenous peroxidase activities were subsequently quenched by using the ready to use solution from the ChromoMAP kit. Then, sections are incubated with primary antibody at the desired dilution in Dako antibody diluent for 1 hour either at room temperature (for pMAPK Ab) or 37° C. for the pAKT antibody. Corresponding negative controls are incubated with AbD only. Sections are subsequently stained using the polymer based amplification system (OmniMAP anti-Rabbit kit) for 4 min at room temperature, followed by 8 min treatment with DAB. Counterstaining of sections is done for 4 min at room temperature with the hematoxylin solution from the ChromoMAP kit. The slides are then dehydrated in graded ethanol and xylene solutions and are mounted with Pertex mounting medium.

### Results:

As shown in FIG. 6, the immunohistochemistry data shows that treatment with Compound A strongly inhibits pAKT, that treatment with Compound B strongly inhibits pMAPK and that treatment with the combination of Compound A and Compound B results in inhibition of both, pAKT and pMAPK.

### Example 4

### Clinical Study with Combination of Compound B and Compound A

| | |
|---|---|
| Title | A phase Ib, open-label study of oral compound B in combination with |
| | oral compound A in adult patients with select advanced solid tumors |
| Brief title | Study of efficacy and safety of the combination of compound B and |
| | compound A in advanced solid tumors |
| Sponsor and Clinical | Novartis, phase Ib |
| Phase | |
| Investigation type | Drug |
| Study type | Interventional |
| Purpose and rationale | To characterize the safety of the combination of Compound B and |
| | Compound A in patients with PIK3CA mutant tumors with or without |
| | genetic alterations in FGFR; study of the combination is based on |
| | clinical and pre-clinical evidence that the co-occurrence of these |
| | molecular alterations may be involved in tumorigenesis or acquisition |
| | of resistance |
| Primary Objective(s) | Objective: To determine the maximum tolerated dose (MTD) and/or |
| and Key Secondary | recommended dose for expansion (RDE) of Compound B in |
| Objective | combination with Compound A |
| Secondary Objectives | Objective 1: To characterize the safety and tolerability of Compound B |
| | in combination with Compound A at the MTD and/or RDE |
| | Objective 2: To determine the single and multiple dose PK profiles of |
| | the combination. |
| | Objective 3: To assess any preliminary antitumor activity of the |
| | combination of Compound B with Compound A |
| Study design | The study includes a phase Ib dose escalation portion to define the |
| | MTD/RP2D for the combination of Compound B and Compound A, |
| | followed by an expansion at the MTD/RDE to further characterize the |
| | safety and efficacy of the combination. |
| Population | Patients ≧ 18 years of age with metastatic or advanced solid tumors |
| | that express PIK3CA mutations with or without genetic alterations in |
| | FGFR. At least 50 patients will be enrolled, of which at least 15 will be |
| | enrolled to the dose escalation part and 35 will be enrolled to the dose |
| | expansion part. |
| Inclusion criteria | Selected inclusion criteria: |
| | Histologically/cytologically confirmed advanced or metastatic solid |
| | tumors who have failed standard therapy or for whom no effective |
| | standard anti-cancer therapy exists |
| | Documented PIK3CA mutations in all patients in dose escalation |
| | and expansion with or without documented genetic alterations in |
| | FGFR depending upon dose expansion cohort (either local or central |
| | determination) |
| | Measurable or evaluable disease defined by RECIST v1.1 |
| | ECOG performance status of ≦2 |
| Exclusion criteria | Selected exclusion criteria: |
| | Prior PI3Ki or selective FGFR or MEK inhibitor treatment (for |
| | patients enrolled to expansion part) |
| | Colorectal cancer (for patients enrolled to expansion part) |
| | Patients with diabetes mellitus requiring insulin treatment and/or with |
| | clinical signs or with fasting glucose ≧ 140 mg/dL/7.8 mmol/L, history |
| | of clinically significant gestational diabetes mellitus or documented |
| | steroid-induced diabetes mellitus |
| | Use of medications that increase serum levels of phosphorus and/or |
| | calcium |
| | Inorganic phosphorus outside of normal limits |
| | Total and ionized serum calcium outside of normal limits |
| Investigational and | The investigational treatments to be used in this study are Compound |
| reference therapy | B and Compound A. |
| Efficacy assessments | Tumor response as per RECIST version 1.1 |
| | Progression free survival |
| | Best overall response |
| | Overall response rate |
| Safety assessments | Incidence rate of Dose Limiting Toxicities (DLT) during the first cycle |
| | of Compound B and Compound A combination treatment |
| | Adverse events and serious adverse events, changes in laboratory |
| | values, assessments of physical examinations, vital signs, |
| | electrocardiograms, and dose interruptions, dose reduction, and dose |
| | intensity. |
| Other assessments | Compound B and Compound A pharmacokinetic evaluations |
| | Gene alteration/expression profiles (e.g. at baseline, relapse) in tumor |
| | tissue |
| Data analysis | A Bayesian logistic regression model will guide the dose escalation to |
| | determine the MTD(s)/RDE |
| Key words | Compound B and Compound A, PI3K inhibitor, FGFR, solid tumor, |
| | metastatic |

This is the first combination trial of BGJ398 with another investigational agent.

This is an open label, phase lb study. The design of the dose escalation part of the study was chosen in order to establish the dose of Compound B (not to exceed 125 mg) that can safely be combined with up to 400 mg qd of Compound A in patients whose tumors harbor PIK3CA mutations. The dose escalation will be guided by a Bayesian logistic regression model (BLRM).

The current open-label phase lb dose escalation study design using a BLRM is a well-established method to estimate the MTD and/or RDE in cancer patients. The adaptive BLRM will be guided by the escalation with overdose control (EWOC) principle to control the risk of DLT in future patients on study. The use of Bayesian response adaptive models for small datasets has been accepted by EMEA and endorsed by numerous publications, and its development and appropriate use is one aspect of the FDA's Critical Path Initiative.

Once the MTD(s)/RDE has been determined, additional patients will be enrolled to one of three expansion arms to assess the anti-tumor activity of the Compound B in combination with Compound A in metastatic breast cancer and other solid tumors with PIK3CA mutations with or without FGFR pathway alterations in addition to continued evaluation of safety.

Objectives are:

To determine the maximum tolerable dose (MTD) of Compound B in combination with compound A (endpoint: Incidence of dose limiting toxicities (DLTs); and further to characterize the safety and tolerability of compound B in combination with compound A at the MTD and/or RDE (endpoint: Incidence and severity of adverse events and serious adverse events, changes in laboratory values, electrocardiograms and vital signs. Dose interruptions, reductions and dose intensity), to determine the single and multiple dose PK profiles of the investigational drugs in combination (Compound B and Compound A) (endpoint: time vs. concentration profiles, derived PK parameters of Compound B and Compound A and known active metabolites), and to assess any preliminary antitumor activity of the combination of Compound B and Compound A. (endpoint: Overall response rate (ORR; CR+PR) assessed by investigators per RECIST ver 1.1, and progression-free survival) (CR=Complete Response, PR=Partial Response). During the course of the study so far the combination appears to be safe and tolerable.

1. A pharmaceutical combination comprising:
(a) a compound of formula (I) 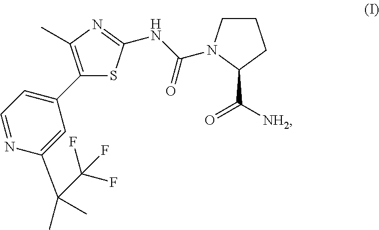 [SMILES: Cc1nc(NC(=O)N2CCC[C@H]2C(N)=O)sc1-c1ccnc(C(C)(C)C(F)(F)F)c1]
or a pharmaceutically acceptable salt thereof; and
(b) a fibroblast growth factor receptor (FGFR) kinase inhibitor, or a pharmaceutically acceptable salt thereof.

2. The pharmaceutical combination according to claim 1, wherein the FGFR kinase inhibitor is selected from the group consisting of TKI258, 3-(2,6-dichloro-3,5-dimethoxy-phenyl)-1-{6-[4-(4-ethylpiperazin-1-l)-phenylamino]-pyrimidin-4-yl}-1-methyl-urea, AZD4547, PD173074, intedanib, dovitinib, brivanib (especially the alaninate), cediranib, masitinib, orantinib, ponatinib, E-7080, HGS1036/FP-1039, MFGR1877S, GP369/AV-396b, and HuGAL-FR21 or a pharmaceutically acceptable salt thereof.

3. The pharmaceutical combination according to claim 1 for simultaneous, separate or sequential use for the treatment of a cancer.

4. The pharmaceutical combination according to claim 3, wherein the cancer is a solid tumor.

5. The pharmaceutical combination according to claim 4, wherein the solid tumor is selected from the group consisting of breast cancer, bladder cancer, endometrial cancer, and ovarian cancer.

6. The pharmaceutical combination according to claim 3, wherein the cancer is mediated by the activity of phosphatidylinositol 3-kinase and/or FGFR kinase.

7-12. (canceled)

13. A method of treating a cancer comprising administering to a subject having said cancer a jointly therapeutically effective amount of a compound of formula (I) 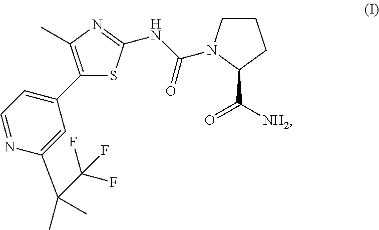 [SMILES: Cc1nc(NC(=O)N2CCC[C@H]2C(N)=O)sc1-c1ccnc(C(C)(C)C(F)(F)F)c1]
or a pharmaceutically acceptable salt thereof; and a fibroblast growth factor receptor (FGFR) kinase inhibitor.

14. The method according to claim 13, wherein the FGFR kinase inhibitor is selected from the group consisting of TK1258, 3-(2,6-dichloro-3,5-dimethoxy-phenyl)-1-{6-[4-(4-ethylpiperazin-1-l)-phenylamino]-pyrimidin-4-yl}-1-methyl-urea, AZD4547, PD173074, intedanib, dovitinib, brivanib (especially the alaninate), cediranib, masitinib, orantinib, ponatinib, E-7080, HGS1036/FP-1039, MFGR1877S, GP369/AV-396b, and HuGAL-FR21 or a pharmaceutically acceptable salt thereof.

15. The method according to claim 13, wherein the cancer is a solid tumor.

16. The method according to claim 15, wherein the solid tumor is selected from the group consisting of breast cancer, bladder cancer, endometrial cancer, and ovarian cancer.

17. The method according to claim 13, wherein the cancer is mediated by the activity of phosphatidylinositol 3-kinase and/or FGFR kinase.

18. A pharmaceutical composition comprising the pharmaceutical combination according to claim 1 and optionally at least one pharmaceutically acceptable carrier.

19. A kit comprising a compound of formula (I) according to claim 1 or a pharmaceutically acceptable salt thereof, and a package insert or label providing instruction for treating a cancer by co-administering at least one FGFR kinase inhibitor.

20. Use PIK3CA amplification or somatic activating mutation and FGFR1, FGFR2, FGFR3 or FGFR4 amplification or somatic activating mutations as biomarkers to select patients likely responding to the treatment of the pharmaceutical combination of the invention according to claim 1.

21. The use of claim 20, wherein the gene amplification is detected by fluorescence in site hybridization (FISH).
